(19) United States
(12) Patent Application Publication
(10) Pub. No.: US 2010/0069635 A1
(43) Pub. Date: Mar. 18, 2010

(54) ROSUVASTATIN DEHYDROABIETYLAMINE SALT

(75) Inventors: Satyanarayana Bollikonda, Hyderabad (IN); Sridhar Chaganti, Hyderabad (IN); Ranga Reddy Tamma, Hyderabad (IN); Loka Maheshwari Pochaiah h Dommati, Secunderabad (IN)

Correspondence Address: DR. REDDY''S LABORATORIES, INC., 200 SOMERSET CORPORATE BLVD, SEVENTH FLOOR, BRIDGEWATER, NJ, 08807-2862, US

(73) Assignees: DR. REDDY'S LABORATORIES LTD., Hyderabad 500016 Andhra Pradesh (IN); DR. REDDY'S LABORATORIES, INC., Bridgewater, NJ (US)

(21) Appl. No.: 12/516,846

(22) PCT Filed: Nov. 29, 2007
(86) PCT No.: PCT/US2007/085888

§ 371 Date: May 29, 2009

Related U.S. Application Data
(60) Provisional application No. 60/891,256, filed on Feb. 23, 2007.

(30) Foreign Application Priority Data
Nov. 29, 2006 (IN) 2216/CHE/2006

(51) Int. Cl. C07D 239/02

(52) U.S. Cl. 544/332

(57) ABSTRACT

There is provided a compound, which is dehydroabietylamine salt of rosuvastatin. Also provided are processes for making rosuvastatin calcium that include formation of dehydroabietylamine salt of rosuvastatin.

### TECHNICAL FIELD

The present patent application relates to rosuvastatin dehydroabietylamine salt and a process for its preparation. It also relates to a process for the preparation of purified rosuvastatin and its salts.

### INTRODUCTION OF THE APPLICATION

Rosuvastatin calcium is a synthetic lipid-lowering agent, which acts by inhibiting 3-hydroxy-3-methylglutaryl-coenzyme A (HMG-CoA) reductase. This enzyme catalyzes conversion of HMG-CoA to mevalonate, an early and rate-limiting step in cholesterol biosynthesis.

Rosuvastatin calcium is chemically described as bis[(E)-7-[4-(4-fluorophenyl)-6-isopropyl-2-N-[methyl-N-(methylsulfonyl)amino]pyrimidin-5-yl]-(3R,5S)-3,5-dihydroxy hept-6-enoic acid] calcium salt, and is represented by the structural Formula I:

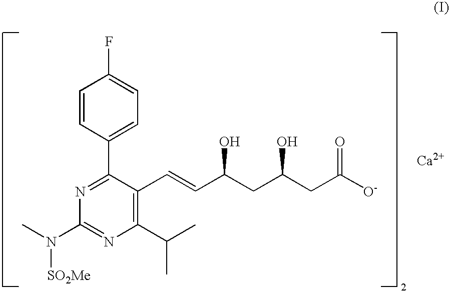

Rosuvastatin calcium is commercially available under the brand name CRESTOR as tablets containing 5, 10, 20 or 40 mg of active ingredient.

U.S. Re-issue patent No. RE 37314 E describes rosuvastatin and its derivatives along with its salts and also gives a process for the preparation of rosuvastatin calcium form its sodium salt.

PCT Application Publication Nos. WO2001/60804, WO2005/051921, WO2005/077916 disclose various salts of rosuvastatin other than the sodium salt and their use in the preparation of rosuvastatin calcium.

There is a continued need for additional processes of making rosuvastatin and its salts.

### SUMMARY

In one aspect, there is provided a compound, which is dehydroabietylamine salt of rosuvastatin having the formula:

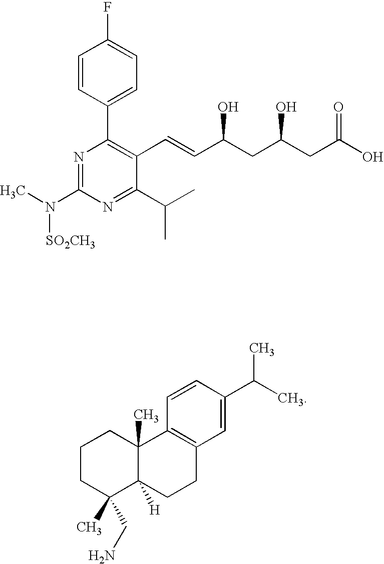

Various embodiments and variants are provided.

In another aspect, there is provided a process of making calcium salt of rosuvastatin, the process including:

a) providing a solution or suspension of free acid of rosuvastatin;

b) treating the solution or suspension with dehydroabietylamine in the amount sufficient to convert the free acid to a dehydroabietylamine salt of rosuvastatin; and

c) converting the dehydroabietylamine salt of rosuvastatin to the calcium salt of rosuvastatin. Various embodiments and variants are provided.

In yet another aspect, there is provided a process for preparation of calcium salt of rosuvastatin, the process including:

a) providing a solution or suspension of dehydroabietylamine salt of rosuvastatin in an organic solvent;

b) treating the solution or suspension with an alkali base to form an alkali salt of rosuvastatin; and

c) treating alkali salt of rosuvastatin with a source of calcium cations to obtain the calcium salt of rosuvastatin. Various embodiments and variants are provided.

### BRIEF DESCRIPTION OF THE DRAWINGS

FIG. 1 is an XRPD pattern for a sample of crystalline rosuvastatin dehydroabietylamine salt of Formula II obtained according to a process described in Example 4.

FIG. 2 is an XRPD pattern for a sample of rosuvastatin calcium obtained according to a process described in Example 5.

### DETAILED DESCRIPTION

Non-calcium salts of rosuvastatin may provide a method for purifying rosuvastatin through crystallization. The present patent application provides a crystalline salt of rosuvastatin and a process for its preparation, which allows for the purification of rosuvastatin that may be subsequently converted to its calcium salt.

As set forth above, dehydroabietylamine salt of rosuvastatin is provided. The dehydroabietylamine salt of rosuvastatin may be in the form of anhydrous solid, solvate, hydrate, crystalline, or amorphous solid. In an embodiment, the rosuvastatin dehydroabietylamine salt is in a crystalline form, which may be characterized by its X-ray powder diffraction (“XRPD”) pattern. The crystalline form of dehydroabietylamine salt of rosuvastatin has significant peaks at about 3.7, 6, 7.5, 11.3, 12.1, 12.5, and 21.8, ±0.2 degrees two theta. Preferably, at least two of these characterizing peaks are present in the XRPD pattern. The pattern may be also characterized by additional XRPD peaks at about 14.7 and 21.3, ±0.2 degrees 2θ. FIG. 1 illustrates an example of an XPRD pattern for the crystalline dehydroabietylamine salt of rosuvastatin. The XRPD data reported herein were obtained using Cu Kα radiation, having the wavelength 1.541 Å and using a Bruker AXS D8 Advance Powder X-ray Diffractometer.

Also provided is a process for preparation of rosuvastatin dehydroabietylamine salt that includes the steps of:

a) providing a solution or suspension of free acid of rosuvastatin;

b) treating the solution or suspension with dehydroabietylamine in the amount sufficient to convert the free acid to a dehydroabietylamine salt of rosuvastatin; and

c) converting the dehydroabietylamine salt of rosuvastatin to the calcium salt of rosuvastatin.

Each step is separately contemplated.

Step a) involves providing a solution or suspension of free acid of rosuvastatin in a solvent. The solution or suspension of free acid of rosuvastatin may be obtained by dissolving or suspending rosuvastatin in a solvent, or may be obtained in situ, directly from the reaction in which rosuvastatin is formed. In one variant, the providing step a) includes i) synthesizing the free acid of rosuvastatin in situ, ii) separating a solution of the free acid of rosuvastatin as an organic phase from the reaction mixture of the step i), and iii) using the separated solution of the step ii) directly in said step b) without isolating the free acid.

The starting mass is a solution or a suspension depending on the choice of the solvent. Temperatures which are adopted for preparation of the mixture may range from about 25° C. to about 100° C. Temperatures greater than 100° C. are also contemplated.

Solvents which may be used for dissolving or suspending rosuvastatin include, but are not limited to, nitriles such as acetonitrile and propionitrile; alcohols, such as methanol, ethanol, isopropyl alcohol, and n-propanol; ketones, such as acetone, ethyl methyl ketone, and methyl isobutyl ketone; esters such as ethyl acetate, n-propyl acetate, n-butyl acetate, and t-butyl acetate; ethers, such as diethyl ether, dimethyl ether, diisopropyl ether, and 1,4-dioxane, hydrocarbons such as toluene, xylene, n-heptane, cyclohexane, n-hexane; or mixtures thereof or their combinations with water. The preferred solvent is acetonitrile. The quantity of solvent used depends on the solvent and on the dissolution temperature adopted when it is in the form of a solution. The concentration of rosuvastatin in the solution may generally range from about 0.1 to about 10 g/ml in the solvent.

When the starting mass is in the form of a solution, it may be filtered to remove the undissolved particles followed by further processing. The undissolved particles may be removed, for example, by filtration, centrifugation, decantation, and other techniques. The solution is filtered by passing through paper, glass fiber, or other membrane material, or a clarifying agent such as celite. Depending upon the equipment used and the concentration and temperature of the solution, the filtration apparatus may need to be preheated to avoid premature crystallization.

In step b), the solution or suspension of rosuvastatin is treated with dehydroabietylamine, which may be chemically described as 1,4a-dimethyl-7-isopropyl-1,2,3,4a,9,10a-octahydro-1-phenantherene methyl amine and is represented by the Formula VII:

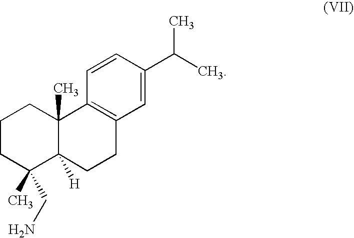

Dehydroabietylamine that is used for the preparation of compound of Formula II may be racemic or in a diasteriomerically pure form.

Preferably, the amount of dehydroabietylamine is sufficient to convert the free acid to the amine salt. For example, from about 0.5 moles to about 5.0 moles of dehydroabietylamine may be used per 1 mole of rosuvastatin, more preferably, 1.05 mole to 1.5 mole of amine per mole of rosuvastatin. The dehydroabietylamine may be added at temperatures ranging from about 20° C. to about 70° C., or at lower temperatures in the range of from about −10° C. to about 20° C. The dehydroabietylamine may be used in the form of a solid or in the form of its aqueous solution or in the form of its solution in an organic solvent.

As each step is separately contemplated, the process may begin with an isolated dehydroabietylamine salt of rosuvastatin, which is then provided in a solvent for further processing, or the dehydroabietylamine salt of rosuvastatin formed as described above may be used directly after step b).

When a solution is prepared by dissolving dehydroabietylamine salt of rosuvastatin in a suitable solvent, any form of rosuvastatin dehydroabietylamine salt such as any crystalline form including solvates and hydrates may be utilized. Solvents which are used for preparing the solution include, but are not limited to, alcohols such as methanol, ethanol, isopropanol, n-butanol; ethers, such as diethyl ether, diisopropyl ether, tetrahydrofuran; nitrile solvents, such as acetonitrile, propionitrile; lower carboxylic acids solvents such as acetic acid; and mixtures thereof.

In an embodiment, dehydroabietylamine salt of rosuvastatin is isolated and further purified by recrystallization or slurrying, or a combination thereof. Solvents which are useful for recrystallization or slurry include, but are not limited to, nitriles such as acetonitrile, propionitrile; alcohols such as methanol, ethanol, propanol, isopropanol; halogenated hydrocarbons such as dichloromethane, ethylene dichloride, chloroform, hydrocarbons such as toluene, xylene, n-hexane, n-heptane, cyclohexane; esters such as ethyl acetate, propyl acetate; ethers such as diethyl ether, diisopropyl ether, methyl tertiary-butyl ether or mixtures thereof in various proportions. The preferred crystallization medium is a mixture of nitrile solvent and an alcoholic solvent. In an embodiment, purification is carried out by recrystallization in a combination of a nitrile solvent and an alcoholic solvent in a ratio of 1:1.

In step c), the dehydroabietylamine salt of rosuvastatin is converted to the calcium salt of rosuvastatin. Preferably, the converting step involves treating the dehydroabietylamine salt of rosuvastatin with an alkali base to obtain an alkali salt of rosuvastatin and treating the resulting intermediate alkali salt of rosuvastatin with a source of calcium cations. Non-limiting examples of alkali bases include lithium hydroxide, sodium hydroxide, sodium carbonate, sodium bicarbonate, potassium hydroxide, potassium carbonate, and potassium bicarbonate. Sodium hydroxide is preferred. These bases may be used in the form of solids or in the form of aqueous solutions. For example, aqueous solutions containing from about 5% to about 50%, or about 10% to 20%, (w/v) of the corresponding base is used. The intermediate base addition salt is in an aqueous solution and may be further made free of its organic impurities by washing the aqueous layer with a less polar solvents like esters such as ethyl acetate, n-propyl acetate, n-butyl acetate, t-butyl acetate; ethers such as diethyl ether, diisopropyl ether; hydrocarbons such as toluene, xylene, n-heptane, cyclohexane, n-hexane.

The intermediate alkali salt is then converted to calcium salt of rosuvastatin by a treatment with a source of calcium cations. The calcium ions may be generated, for example, by using calcium compounds such as calcium chloride, calcium hydroxide, calcium carbonate, calcium acetate, calcium sulfate, calcium borate, calcium tartarate, calcium bromide, or other compound capable of generating calcium ion. The preferred sources of calcium ions are calcium chloride and hydrates of calcium chloride.

Rosuvastatin calcium may be isolated by maintaining the reaction mixture at temperatures of from below about 10° C. to about 25° C., for a period of time required for a more complete formation of the solid product. Isolation may be enhanced by methods such as cooling, partial removal of the solvent from the mixture, or by adding an anti-solvent to the reaction mixture or a combination thereof. The method by which the solid material is recovered from the final mixture, with or without cooling below the operating temperature, can be any of techniques such as filtration by gravity, or by suction, centrifugation. The crystals so isolated will carry a small proportion of occluded mother liquor containing a higher percentage of impurities. If desired the crystals are washed on the filter with a solvent to wash out the mother liquor. The wet cake obtained in step c) may optionally be further dried. Drying is carried out in a tray dryer, vacuum oven, air oven, fluidized bed drier, spin flash dryer, flash dryer. Drying is carried out at temperatures of about 35° C. to about 70° C., and is carried out for periods ranging from about 1 to about 20 hours.

While the invention is not limited to the specific embodiments described, the synthetic scheme for a preferred variant of the process described herein may be illustrated as follows:

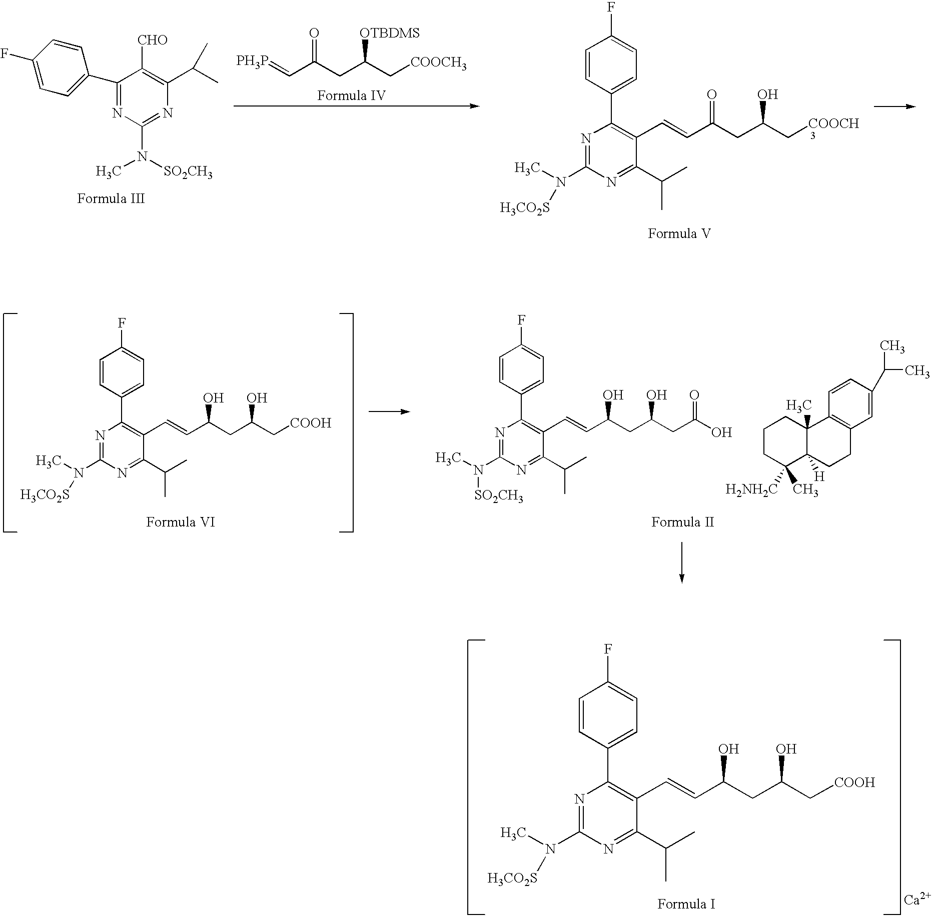

Starting rosuvastatin for the purpose of use in the processes described herein may be prepared by known processes in the art, or by a process that includes:

a) reaction of N-[4-(4-fluorophenyl)-6-isopropyl-2-(N-methyl-N-methylsulfonylamino)] pyramidine carboxaldehyde of Formula III with less than about 1.5 equivalents of methyl (3R)-3-(tert-butyl dimethyl silyloxy)-5-oxo-6-triphenyl-phosphoranylidene hexanoate of Formula IV under suitable reaction conditions, followed by deprotection of the hydroxy group to give methyl-7-[4-(4-fluorophenyl)-6-isopropyl-2-(N-methyl-N-methylsulphonylamino) pyrimidin-5-yl]-(3R)-3-hydroxy-5-oxo-(E)-6-heptenate of Formula V.

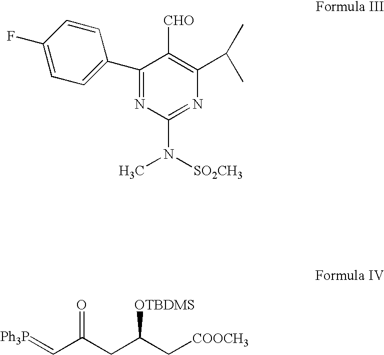

b) reaction of methyl-7-[4-(4-fluorophenyl)-6-isopropyl-2-(N-methyl-N-methylsulphonylamino) pyrimidin-5-yl]-(3R)-3-hydroxy-5-oxo-(E)-6-heptenate of Formula V with a suitable reducing agent in the presence of a chelating agent at a temperature of lower than about −80° C. followed by hydrolysis of the ester obtained to give rosuvastatin of Formula VI.

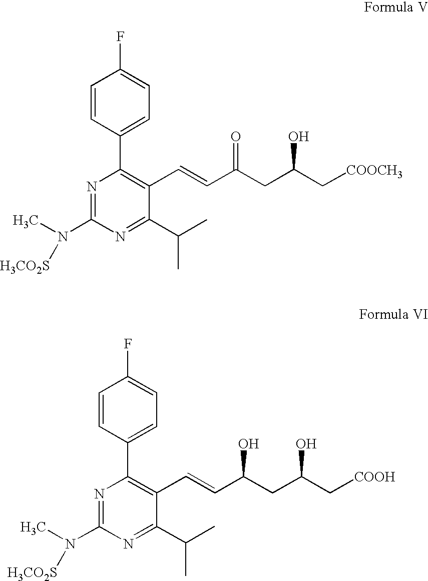

The mole ratio of methyl (3R)-3-(tert-butyl dimethyl silyloxy)-5-oxo-6-triphenyl-phosphoranylidene hexanate of Formula IV used is less than about 1.5 to less than about 1.25 which results in better economy of the process. Solvents which are useful for the reaction include hydrocarbons such as toluene, xylene, n-heptane, cyclohexane, n-hexane; nitriles such as acetonitrile, propionitrile; or mixtures thereof or their combinations with water in various proportions.

Deprotection is carried out using acids such as hydrochloric acid, hydrobromic acid, or hydrofluoric acid. Temperatures for conducting the reaction range from about 10 to about 200° C. or from about 30° C. to about 150° C. Solvents which may be used for the deprotection reaction include, but are not limited to, hydrocarbons such as toluene, xylene, n-heptane, cyclohexane, n-hexane; nitriles such as acetonitrile, propionitrile; or mixtures thereof or their combinations with water in various proportions.

The compound of Formula V may be isolated using techniques such as trituration, slurry or recrystallization in a suitable solvent. Solvents which are include, ketones such as acetone, ethyl methyl ketone, methyl isobutyl ketone; hydrocarbons such as toluene, xylene, n-heptane, cyclohexane, n-hexane; esters such as ethyl acetate, n-propyl acetate, n-butyl acetate, t-butyl acetate; ethers such as diethyl ether, dimethyl ether, diisopropyl ether; nitriles such as acetonitrile, propionitrile; or mixtures thereof or their combinations with water in various proportions.

Reducing agents which are useful for reduction include, but are not limited to sodium borohydride and lithium borohydride. The role of the chelating agent is to form a chelate complex with the compound of Formula V and to direct the reduction to take place at the desired location. Chelating agents which are used include, but are not limited to diethyl methoxy borane, diethyl ethoxy borane. Solvents which are used include, but are not limited to protic solvents; alcohols such as methanol, ethanol, isopropanol, n-butanol; ethers such as diethyl ether, diisopropyl ether, tetrahydrofuran; nitrile solvents such as acetonitrile, propionitrile; and mixtures thereof. The reduction reaction is carried out at temperatures lower than about −80° C. or lower than about −90° C. to avoid the formation of the diastereomeric impurity in the reaction mass.

Rosuvastatin or its salts prepared as described herein preferably have purity greater than 98%, more preferably greater than about 99%, yet more preferably, greater than about 99.5%, and contain less than about 0.5%, or less than about 0.1% of the corresponding impurities like the lactone impurity and the diastereomeric impurity, both described below, as characterized by a high performance liquid chromatography (“HPLC”) chromatogram obtained from a mixture containing the desired compound and one or more of the impurities. The percentage here refers to the area-% of the peaks representing the impurities as measured by the HPLC method set forth herein below.

The diastereomeric impurity, which may be described chemically as (+)-(3R,5R)-7[4-(4-Fluorophenyl)-6-isopropyl-2-(N-methyl-N-methanesulfonylamino)pyrimidin-5-yl]-3,5-dihydroxy-6-(E)-heptenoic acid, is represented by the Formula Ia:

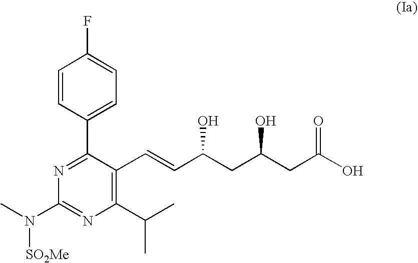

Preferably, rosuvastatin or its salts prepared as described herein contain from about 0.01% to about 0.1% of this diastereomeric impurity.

As used herein “lactone impurity of rosuvastatin” refers to N-(4-(4-Fluoro phenyl)-5-[2(4-hydroxy-6-oxo-tetrahydro-pyran-2-yl)-vinyl]-6-isopropyl-pyrimidin-2-yl)-N-methyl-methanesulfonamide represented by Formula Ib:

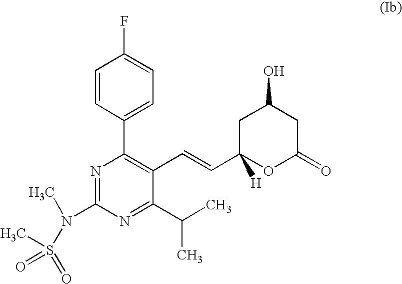

In an embodiment, the present patent application also provides pharmaceutical compositions that include rosuvastatin or its salts prepared according to the processes described herein along with one or more pharmaceutically acceptable carriers, excipients or diluents

The pharmaceutical compositions of rosuvastatin or its pharmaceutically acceptable salts along with one or more pharmaceutically acceptable carriers of this invention may further formulated as: solid oral dosage forms such as, but not limited to, powders, granules, pellets, tablets, and capsules; liquid oral dosage forms such as but not limited to syrups, suspensions, dispersions, and emulsions; and injectable preparations such as but not limited to solutions, dispersions, and freeze dried compositions. Formulations may be in the form of immediate release, delayed release or modified release. Further, immediate release compositions may be conventional, dispersible, chewable, mouth dissolving, or flash melt preparations, and modified release compositions that may comprise hydrophilic or hydrophobic, or combinations of hydrophilic and hydrophobic, release rate controlling substances to form matrix or reservoir or combination of matrix and reservoir systems. The compositions may be prepared by direct blending, dry granulation or wet granulation or by extrusion and spheronization. Compositions may be presented as uncoated, film coated, sugar coated, powder coated, enteric coated or modified release coated. Compositions of the present invention may further comprise one or more pharmaceutically acceptable excipients.

Pharmaceutically acceptable excipients that find use in the present invention include, but are not limited to: diluents such as starch, pregelatinized starch, lactose, powdered cellulose, microcrystalline cellulose, dicalcium phosphate, tricalcium phosphate, mannitol, sorbitol, sugar; binders such as acacia, guar gum, tragacanth, gelatin, polyvinyl pyrrolidone, hydroxypropyl cellulose, hydroxypropyl methylcellulose, pregelatinized starch; disintegrants such as starch, sodium starch glycolate, pregelatinized starch, crospovidone, croscarmellose sodium, colloidal silicon dioxide; lubricants such as stearic acid, magnesium stearate, zinc stearate; glidants such as colloidal silicon dioxide; solubility or wetting enhancers such as anionic or cationic or neutral surfactants; complex forming agents such as various grades of cyclodextrins, resins; release rate controlling agents such as hydroxypropyl cellulose, hydroxymethyl cellulose, hydroxypropyl methylcellulose, ethyl cellulose, methyl cellulose, various grades of methyl methacrylates, waxes. Other pharmaceutically acceptable excipients that are of use include but are not limited to film formers, plasticizers, colorants, flavoring agents, sweeteners, viscosity enhancers, preservatives, antioxidants.

In the compositions of present invention rosuvastatin or its pharmaceutically acceptable salts is a useful active ingredient in the range of 0.5 mg to 100 mg, or 1 mg to 50 mg.

Certain specific aspects and embodiments of this invention are described in further detail by the examples below, which are provided only for the purpose of illustration and are not intended to limit the scope of the invention in any manner.

### EXAMPLE 1

### Preparation of Methyl-7-[4-(4-Fluorophenyl)-6-Iso-Propyl-2-(N-Methyl-N-Methylsulphonylamino) Pyrimidin-5-Yl]-(3R)-3-Hydroxy-5-Oxo-(E)-6-Heptenate (Formula V)

Methyl (3R)-3-(tert-butyl dimethyl silyloxy)-5-oxo-6-triphenyl-phosphoranyllidene hexanoate (10 g), N-[4-(4-Fluoro-phenyl)-5-formyl-6-isopropyl-pyrimidin-2-yl]-N-methyl-methanesulfonamide (6.9 g), toluene (100 ml) were taken into a round bottom flask and heated to about 110° C. The reaction mass was maintained at the same temperature for about 24 hours. Reaction completion was checked using thin layer chromatography. After the reaction was completed, the reaction mass was distilled at a temperature of about 45° C. under low pressure. To the residue obtained, a 1:9 mixture of ethyl acetate and n-hexane (100 ml) was added and stirred at about 26° C. for about 1 hour. The mixture was then cooled to about 10° C. and maintained under stirring for another 1 hour. The separated solid was filtered, and the filtrate was distilled off completely under high vacuum at about 45° C. To the residue obtained, acetonitrile (130 ml) was added at about 26° C., and then further cooled to about 5° C. The mixture was maintained at 5° C. for about 15 minutes and then a solution of 48% aqueous hydrogen fluoride in acetonitrile (13 ml) was added to it and maintained at the same temperature for about 1 hour. The reaction mass was then allowed to heat to 26° C. and maintained for about 1 hour. Reaction completion was checked using thin layer chromatography. After the reaction was completed, saturated sodium bicarbonate solution (140 ml) was added to the reaction mass until pH was adjusted to 7.0. Dichloromethane (300 ml) was added to the above reaction mass and stirred for about 10 minutes. The organic layer was separated and the aqueous layer was extracted into dichloromethane (100 ml). The combined dichloromethane layer was distilled off at about 39° C. under high vacuum to give 11.2 g of the title compound.

### EXAMPLE 2

### Preparation of Methyl-7-[4-(4-Fluorophenyl)-6-Iso-Propyl-2-(N-Methyl-N-Methylsulphonylamino) Pyrimidin-5-YL]-(3R)-3-Hydroxy-5-Oxo-(E)-6-Heptenate (Formula V)

Methyl (3R)-3-(tert-butyl dimethyl silyloxy)-5-oxo-6-triphenyl-phosphoranyllidene hexanate (100 g), N-[4-(4-fluoro-phenyl)-5-formyl-6-isopropyl-pyrimidin-2-yl]-N-methyl-methanesulfonamide (69 g), acetonitrile (1000 ml) were taken into a round bottom flask and heated to about 80° C. The reaction mass was maintained at the same temperature for about 24 hours. Reaction completion was checked using thin layer chromatography. After the reaction was completed, the reaction mass was distilled at a temperature of about 48° C. under reduced pressure. To the residue obtained (175 g), acetonitrile (875 ml) was added and stirred at about 26° C. for about 1 hour. The mixture was then cooled to about 5° C. and a solution of 48% aqueous hydrogen fluoride (36 ml) was added to it. After the addition was completed, the reaction mass was then allowed to heat to about 26° C. and maintained for about 1 hour. After the reaction was completed, saturated sodium bicarbonate solution (610 ml) was added to the reaction mass until the pH was adjusted to 7.0. Dichloromethane (1750 ml) was added to the above reaction mass and stirred for about 10 minutes. The organic layer was separated and the aqueous layer was extracted into dichloromethane (855 ml). The combined dichloromethane layer was washed with saturated sodium chloride solution (885 ml) and then distilled off at about 39° C. under high vacuum to give 145 g of the title compound as a residue. To 8.0 g of the residue obtained, a 9:1 mixture of n-hexane in ethyl acetate (150 ml) was added and stirred at about 25° C. for about 1 hour. The separated solid was filtered and washed with a 9:1 mixture of n-hexane in ethyl acetate (125 ml) to yield 5.5 g of the title compound.

### EXAMPLE 3

### Preparation of Dehydroabeitylamine Salt of Rosuvastatin (Formula II)

Methyl-7-[4-(4-fluorophenyl)-6-isopropyl-2-(N-methyl-N-methylsulphonylamino) pyrimidin-5-yl]-(3R)-3-hydroxy-5-oxo-(E)-6-heptenate (25 g), tetrahydrofuran (750 ml) and methanol (200 ml) were taken into a clean and dry 4 neck round bottom flask and subjected to stirring followed by cooling to a temperature at about −98° C. 1M diethyl methoxy borane (66 ml) in tetrahydrofuran was added to the above reaction mass at a temperature of about of −100° C. under a nitrogen atmosphere for a period of about 1.5 hours to control the formation of the impurities. To the obtained reaction mass sodium borohydride (3 g) was added at the same temperature and the reaction mixture was subjected to stirring for another 1 hour. Reaction completion was checked using thin layer chromatography. After the reaction was completed, acetic acid (35 ml) was added to the above reaction mass at the same temperature and maintained for about 30 minutes. The temperature was slowly raised to about 0° C. and maintained for about 1 hour. Then the temperature was raised to about 5° C. and 10% aqueous sodium bicarbonate solution (600 ml) was added, and ensured that the pH of the reaction mass reached 7.0. Dichloromethane (700 ml) was added to the above reaction mass and stirred for about 15 minutes. The organic layer was separated and the aqueous layer was extracted into dichloromethane (200 ml). The combined dichloromethane layer was washed with saturated sodium chloride solution (300 ml). The dichloromethane layer was then dried over sodium sulphate (95 g) and subjected to distillation at about 45° C. under high vacuum. The residue obtained was co-distilled with methanol (300 ml) in three equal lots at a temperature of about 45° C. under vacuum. To the residue obtained, water (192 ml) was added and subjected to stirring with simultaneous cooling to a temperature of about 10° C. Sodium hydroxide (3.9 g) dissolved in water (15 ml) was added to the above obtained residue at temperature of about 10° C. The reaction mass was then allowed to heat to a temperature of about 30° C. and maintained for about 2 hours. Diisopropyl ether (100 ml) was added to the above reaction mass and subjected to stirring for about 15 minutes. The aqueous layer was separated and washed with diisopropyl ether (2×100 ml).

The obtained aqueous layer was then transferred into a round bottom flask and acetonitrile (120 ml) was added followed by stirring and than cooled to a temperature of about 6° C. 10% aqueous hydrochloric acid (50 ml) was slowly added to the above reaction solution at a temperature of about 6° C. followed by the addition of sodium chloride (30 g). The reaction solution was allowed to settle and the two layers were separated. The acetonitrile layer was dried over sodium sulphate (2 g) and taken into a round bottom flask. The acetonitrile layer was then subjected to cooling to a temperature of about 6° C., then dehydroabietylamine (8.5 g) was added and subjected to stirring for about 1 hour. The separated solid was filtered and washed with acetonitrile (20 ml). The obtained solid was dried at a temperature of about 43° C. under high vacuum for about 5 hours to yield 14.6 g of the title compound.

Purity By HPLC: 96.7%

### EXAMPLE 4

### Purification of Dehydroabeitylamine Salt of Rosuvastatin (Formula II)

Dehydroabeitylamine salt of rosuvastatin (44 g) and a 1:1 mixture of acetonitrile and isopropyl alcohol (220 ml) were taken into a round bottom flask and heated to about 75° C. The mixture was then cooled to about 10 to 15° C. and maintained for about 1 hour. The separated solid was filtered and washed with a 1:1 mixture of acetonitrile and isopropyl alcohol (20 ml). The above recrystallization process was repeated twice with the wet solid obtained using 176 ml of a 1:1 mixture of acetonitrile and isopropyl alcohol. The final solid was washed with a 1:1 mixture of acetonitrile and isopropyl alcohol (20 ml). The wet solid obtained was dried at a temperature of about 45° C. for about 3 hours to yield 21.8 g of the title compound.

Purity By HPLC: 99.29%.

% of Impurity 1 (diastereomeric impurity): 0.08%.

### EXAMPLE 5

### Preparation of Rosuvastatin Calcium from Dehydroabeitylamine Salt of Rosuvastatin (Formula I)

Dehydroabeitylamine salt of rosuvastatin (50 g) and water (250 ml) were taken into a round bottom flask and cooled to about 10° C. A solution of sodium hydroxide flakes (3.9 g) in water (25 ml) was added to the above mixture and stirred at about 25° C. for 2 hours to get a clear solution. The above solution was washed with n-hexane (3×180 ml). The solution was then further washed with diisopropyl ether (2×180 ml). Activated charcoal (5.0 g) was added to the aqueous layer and stirred for about 15 minutes. The solution was then filtered through a celite bed and the bed was washed with water (25 ml). The combined filtrate was distilled at about 45° C. under high vacuum to distill of about 80% of the solvent. To the residue obtained water (125 ml) was added and again filtered though a celite bed and the bed was washed with water (25 ml). The combined filtrate was taken into another round bottom flask and cooled to about 10° C. A solution of calcium chloride dihydrate (5.7 g) in water (25 ml) was added to the above filtrate at the same temperature. The reaction mass was allowed to heat to about 25° C. and maintained for about 1 hour. The separated solid was filtered and washed with water (125 ml). The wet solid was dried at about 45° C. for about 4 hours to yield 28.0 g of the title compound.

Purity By HPLC: 99.8%.

% of Impurity 1 (diastereomeric impurity): 0.14%.

% of Impurity 2 (Rosuvastatin lactone): 0.02%.

### EXAMPLE 6

### Determination of Impurities in Rosuvastatin Calcium

Determining the level of impurities in rosuvastatin calcium using HPLC. The HPLC analysis conditions are as described in Table 1.

TABLE 1
HPLC method for detecting the level of the impurities.

| Column and | Develosil ODS-MG-5 250 × 4.6 mm, 5 μm particle size. | |
|---|---|---|
| Packing: | | |
| Buffer: | 2.722 g of KH₂PO₄ and 3.39 of tetrabutyl ammonium | |
| | hydrogen sulfate salt is dissolved in 1000 ml of | |
| | Milli Q water and 2 ml of triethylamine solution is added | |
| | to it and pH adjusted to 3.0 using H₃PO₄ solution. | |
| Mobile | Mixed buffer and acetonitrile in the ratio 650:350 v/v. | |
| Phase A: | | |
| Mobile | Mixed acetonitrile and water in the ratio 800:200 v/v. | |
| Phase B: | | |
| Gradient: | Time (in minutes) | % B Conc. |
| | 0.01 | 0 |
| | 25 | 0 |
| | 40 | 50 |
| | 45 | 70 |
| | 55 | 70 |
| | 58 | 50 |
| | 60 | 0 |
| | 65 | 0 |

| | |
|---|---|
| Temperature: | 20° C. |
| Injection | 15 μL |
| volume: | |
| Flow rate: | 1.2 mL per minute |
| Wavelength: | 242 nm |
| Diluent: | Acetonitrile |
| Run time: | 65 minutes |

| IMPURITY NAME | RRT |
|---|---|
| Rosuvastatin diastereomeric impurity | 1.05 |
| Rosuvastatin lactone impurity | 1.5 |

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A compound, which is dehydroabietylamine salt of rosuvastatin having the formula:

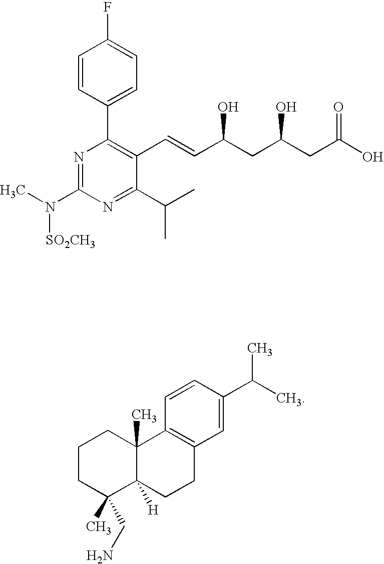

2. The compound of claim 1, which is in solid form.

3. The compound of claim 2, which is in crystalline form.

4. The compound of claim 3, which is characterized by an XRPD pattern having at least two peaks at about 3.7, 6, 7.5, 11.3, 12.1, 12.5, and 21.8, ±0.2 degrees two theta.

5. The compound of claim 1, which has chemical purity of 98% or more as measured by HPLC.

6. The compound of claim 5, which has chemical purity of 99% or more as measured by HPLC.

7. The compound of claim 5, which contains less than about 0.1% of an impurity of the formula Ia:

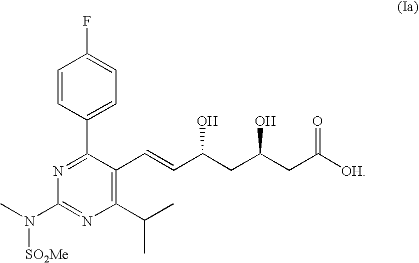

8. The compound of claim 7, which contains more than about 0.01% of the impurity of the formula Ia.

9. A process of making calcium salt of rosuvastatin, said process comprising:
a) providing a solution or suspension of free acid of rosuvastatin;
b) treating said solution or suspension with dehydroabietylamine in the amount sufficient to convert said free acid to a dehydroabietylamine salt of rosuvastatin;
c) converting said dehydroabietylamine salt of rosuvastatin to said calcium salt of rosuvastatin.

10. The process of claim 9, wherein said providing step comprises i) synthesizing said free acid of rosuvastatin, ii) separating a solution of said free acid of rosuvastatin from the reaction mixture of the step i), and iii) using said separated solution of the step ii) directly in said step b) without isolating said free acid of rosuvastatin.

11. The process of claim 9, wherein said amount of said dehydroabietylamine varies from 0.5 mole to 5 mole per mole of said free acid of rosuvastatin.

12. The process of claim 11, wherein said amount of said dehydroabietylamine varies from 1.05 mole to 1.5 mole per mole of said free acid of rosuvastatin.

13. The process of claim 9, wherein said converting step c) comprises A) treating said dehydroabietylamine salt of rosuvastatin with an alkali base to obtain an alkali salt of rosuvastatin; and B) treating said alkali salt of rosuvastatin with a source of calcium cations to obtain said calcium salt of rosuvastatin.

14. The process of claim 13, wherein said source of calcium cations is selected from calcium chloride and hydrates of calcium chloride.

15. The process of claim 9, wherein said dehydroabietylamine salt of rosuvastatin is isolated prior to said converting step c).

16. The process of claim 9, wherein dehydroabietylamine is used in a diasteromerically pure form.

17. The process of claim 15, further comprising recrystallization of the isolated dehydroabietylamine salt of rosuvastatin from a recrystallizing solvent.

18. The process of claim 17, wherein said recrystallizing solvent is a mixture of a nitrile solvent and an alcoholic solvent.

19. The process of claim 9, wherein the solvent of the providing step a) is selected from a nitrile solvent, an alcoholic solvent, a halogenated hydrocarbon, a ketone, an ester, an ethers and a hydrocarbon solvent.

20. The process of claim 9, wherein the solvent of the providing step a) is acetonitrile.

21. The process of claim 18, wherein said organic solvent is selected from a nitrile solvent, an alcoholic solvent and an ether.

22. A process for preparation of calcium salt of rosuvastatin, said process comprising:
a) providing a solution or suspension of dehydroabietylamine salt of rosuvastatin in an organic solvent;
b) treating said solution or suspension with an alkali base to form an alkali salt of rosuvastatin; and
c) treating alkali salt of rosuvastatin with a source of calcium cations to obtain said calcium salt of rosuvastatin.

23. The process of claim 22, wherein said alkali salt obtained in said step b) is not isolated.

24. The process of claim 22, wherein said alkali base is selected from sodium hydroxide and potassium hydroxide.
